(19) United States
(12) Patent Application Publication
(10) Pub. No.: US 2024/0059741 A1
(43) Pub. Date: Feb. 22, 2024

(54) COMPOSITION FOR PREVENTION OR TREATMENT OF SARS-COV-2 INFECTION

(71) Applicant: AJOU UNIVERSITY INDUSTRY-ACADEMIC COOPERATION FOUNDATION, Suwon-si (KR)

(72) Inventors: Hyun Goo WOO, Suwon-si (KR); Masaud SHAH, Cheongju-si (KR); Sung Ung MOON, Yongin-si (KR)

(73) Assignee: AJOU UNIVERSITY INDUSTRY-ACADEMIC COOPERATION FOUNDATION, Suwon-si (KR)

(21) Appl. No.: 18/269,708

(22) PCT Filed: Dec. 24, 2021
(86) PCT No.: PCT/KR2021/019796

§ 371 Date: Jun. 26, 2023

(30) Foreign Application Priority Data
Dec. 24, 2020 (KR) 10-2020-0183488
Jun. 15, 2021 (KR) 10-2021-0077398

(51) Int. Cl. C07K 14/00; A61P 31/14

(52) U.S. Cl. CPC C07K 14/001; A61P 31/14; A61K 38/00

(57) ABSTRACT

The present invention relates to a composition for prevention or treatment of SARS-CoV-2 infection. CSNP1, CSNP2, CSNP3, and CSNP4 bind to receptor the binding domain (RBD) of the spike protein of SARS-CoV-2 to inhibit the interaction of the spike protein of SARS-CoV-2 with ACE2, thereby interfering with the mechanism that SARS-CoV-2 enters cells or evades immunity. Thus, a composition comprising CSNP1, CSNP2, CSNP3, or CSNP4 as an active ingredient is provided as a pharmaceutical agent for prevention or treatment of SARS-CoV-2 infection (COVID19).

### TECHNICAL FIELD

The present disclosure relates to a composition for prevention or treatment of SARS-CoV-2 infection.

### BACKGROUND ART

The pandemic caused by SARS-CoV-2 (COVID-19) has resulted in over 30 million confirmed cases and nearly 900,000 deaths worldwide up to date, posing a serious threat to the health of people worldwide as well as to social stability and economic development. Therefore, many researchers are dedicated to developing technologies that may prepare against SARS-CoV-2, such as those for diagnosis, prevention, and treatment of SARSCoV-2.

It is known that SARS-CoV-2 causes infection in vivo through a mechanism in which a receptor-binding domain part of a spike protein binds to an ACE2 receptor of a host cell. Thus, the present inventors aimed to develop antibodies or antibody-related molecules that bind to the RBD and inhibit the binding of the spike protein of SARS-CoV-2 with the ACE2 receptor of the host cell, thereby preventing infection, treating infectious diseases, and enabling diagnosis eventually.

### DISCLOSURE OF THE INVENTION

### Technical Goals

An object of the present disclosure is to prepare a protein that blocks the binding of a spike protein of SARS-CoV-2 with ACE and provide as a composition for prevention and treatment of SARS-CoV-2 infection, in an attempt to prevent or treat SARS-CoV-2 infection (COVID19) by blocking the interaction of a spike protein, which plays a pivotal role for SARS-CoV-2 to enter the cell and evade immunity of a host, with ACE.

### Technical Solutions

In order to achieve the above object, the present disclosure provides a pharmaceutical composition for prevention or treatment of SARS-CoV-2 infection, including CSNP1, CSNP2, CSNP3, or CSNP4 as an active ingredient.

In addition, the present disclosure provides a co-administration composition for prevention or treatment of SARS-CoV-2 infection, including CSNP1, CSNP2, CSNP3, or CSNP4 as an active ingredient.

In addition, the present disclosure provides a health functional food composition for prevention or alleviation of SARS-CoV-2 infection, including CSNP1, CSNP2, CSNP3, or CSNP4 as an active ingredient.

In addition, the present disclosure provides a prevention or treatment method for SARS-CoV-2 infection, including administering, to a subject, a composition including CSNP1, CSNP2, CSNP3 or CSNP4 as an active ingredient in a therapeutically effective amount.

### Advantageous Effects

According to the present disclosure, CSNP1, CSNP2, CSNP3, and CSNP4 bind to a receptor binding domain (RBD) of a spice protein of SARS-CoV-2 and inhibit interaction of the spice protein of SARS-CoV-2 with ACE2 so as to interfere with a mechanism that SARS-CoV-2 enters cells or evades immunity, such that it is possible to provide a composition including CSNP1, CSNP2, CSNP3, or CSNP4 as an active ingredient as a pharmaceutical agent for prevention or treatment of SARS-CoV-2 infection (COVID19).

### BRIEF DESCRIPTION OF DRAWINGS

FIG. 1 shows diagrams in which a SARS-CoV-2 spike RBD hACE2 interface is analyzed and a CSNP peptide is designed.

FIG. 2 shows results of structural validation of neutralized SARS-CoV-2 spike peptides, CSNPs, using molecular dynamics simulation (MDS).

FIG. 3 shows diagrams of the binding and structural dynamics of CSNP peptides including SARS-CoV-2 spike RBD and hACE2 determined via molecular dynamics simulation (MDS).

FIG. 4 shows results of evaluating an effect of CSNP peptides on interaction of SARS-CoV-2 S1 and hACE2 in hACE2-overexpressed HEK293 cells.

FIG. 5 shows results of analyzing binding dynamics of hACE2, SARS-CoV-2 S1, and CSNP peptides.

FIG. 6 shows results of analyzing binding dynamics of SARS-CoV-2 S1 and al helix peptides (Pep 1˜Pep 5).

FIG. 7 shows results of analyzing a peptide remaining degree through staining with Coomassie brilliant blue dye after treating CSNP1 and CSNP4 with ProtK.

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

The numerical range includes the numerical value defined in the above range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Hereinafter, the present disclosure will be described in more detail.

The present disclosure provides a pharmaceutical composition for prevention or treatment of SARS-CoV-2 infection, including CSNP1, CSNP2, CSNP3, or CSNP4 as an active ingredient.

The CSNP1 has an amino acid sequence represented by SEQ ID NO: 1, the CSNP2 has an amino acid sequence represented by SEQ ID NO: 2, the CSNP3 has an amino acid sequence represented by SEQ ID NO: 3, and the CSNP4 has an amino acid sequence represented by SEQ ID NO: 4.

The CSNP1, CSNP2, CSNP3, or CSNP4 inhibits the interaction of a spice protein of SARS-CoV-2 with ACE2, and the CSNP1, CSNP2, CSNP3, or CSNP4 binds to a receptor binding domain (RBD) of the spice protein of SARS-CoV-2.

The CSNP2 and CSNP3 have lactam rings.

The pharmaceutical composition of the present disclosure may be prepared in a unit dose form or prepared by infusion in a multi-dose container through formulation using pharmaceutically acceptable carriers according to a method that may be easily carried out by a person skilled in the art to which the present disclosure pertains.

The pharmaceutically acceptable carriers are those commonly used in preparation, and include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, but are not limited thereto. The pharmaceutical composition of the present disclosure may further include lubricants, wetting agents, sweetening agents, flavoring agents, emulsifying agents, suspending agents, and preservatives, in addition to the above components.

In the present disclosure, the content of additives included in the pharmaceutical composition is not particularly limited and may be appropriately adjusted within the content range used for conventional preparation.

The pharmaceutical composition may be formulated in one or more external preparation forms selected from the group consisting of injectable formulations such as aqueous solutions, suspensions, and emulsions, pills, capsules, granules, tablets, creams, gels, patches, sprays, ointments, emplastrum agents, lotions, liniments, pastas, and cataplasmas.

The pharmaceutical composition of the present disclosure may include pharmaceutically acceptable carriers and diluents, which are additional for formulation. The pharmaceutically acceptable carrier and diluent include excipients such as starch, sugar, and mannitol, fillers and extenders such as calcium phosphate, cellulose derivatives such as carboxymethylcellulose and hydroxypropyl cellulose, binders such as gelatin, alginate, and polyvinylpyrrolidone, lubricants such as talc, calcium stearate, hydrogenated castor oil, and polyethylene glycol, disintegrants such as povidone and crospovidone, and surfactants such as polysorbates, cetyl alcohol, and glycerol, but are not limited thereto. The pharmaceutically acceptable carrier and diluent may be biologically and physiologically compatible with subjects. Examples of the diluent may include saline, aqueous buffers, solvents, and/or dispersion media, but are not limited thereto.

The pharmaceutical composition of the present disclosure may be administered orally or parenterally (e.g., intravenously, subcutaneously, intraperitoneally, or topically) depending on a desired method. For oral administration, the pharmaceutical composition may be formulated as tablets, troches, lozenges, aqueous suspensions, oily suspensions, prepared powder, granules, emulsions, hard capsules, soft capsules, syrups, or elixirs. For parenteral administration, the pharmaceutical composition may be formulated as injections, suppository agents, powder for respiratory inhalation, aerosols for sprays, ointments, powder for application, oil, and creams.

The dosage range of the pharmaceutical composition of the present disclosure may vary depending on the patient's condition, body weight, age, sex, health status, dietary constitution specificity, the nature of preparations, the degree of diseases, administration duration of a composition, administration methods, administration periods or intervals, excretion rate, and drug forms, and may be appropriately selected by those skilled in the art. For example, the dosage may be in the range of about 0.1 to 10,000 mg/kg but is not limited thereto, while it may be administered in divided doses from one to several times a day.

The pharmaceutical composition may be administered orally or parenterally (e.g., intravenously, subcutaneously, intraperitoneally, or topically) depending on a desired method. A pharmaceutically effective amount and effective dosage of the pharmaceutical composition of the present disclosure may vary depending on formulation methods, administration methods, administration duration, and/or administration routes of the pharmaceutical composition, and those skilled in the art may easily determine and prescribe the dosage effective for desired treatment. Administration of the pharmaceutical composition of the present disclosure may be conducted once a day or several times in divided doses.

In addition, the present disclosure provides a co-administration composition for prevention or treatment of SARS-CoV-2 infection, including CSNP1, CSNP2, CSNP3, or CSNP4 as an active ingredient.

In addition, the present disclosure provides a health functional food composition for prevention or alleviation of SARS-CoV-2 infection, including CSNP1, CSNP2, CSNP3, or CSNP4 as an active ingredient.

The present disclosure may be generally used as a commonly used food product.

The food composition of the present disclosure may be used as a health functional food. The term “health functional food” as used herein refers to food manufactured and processed with raw materials or components having useful functionality for the human body in accordance with the Health Functional Food Act, and the term “functionality” as used herein refers to the intake to derive effectiveness in health care such as regulation of nutrients or physiological actions for the structure and function of the human body.

The food composition of the present disclosure may include common food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents. The food composition of the present disclosure may be manufactured and processed in the form of tablets, capsules, powder, granules, liquids, and pills.

For example, hard capsule preparations among health functional foods in the form of capsules may be prepared by mixing and filling the composition according to the present disclosure in conventional hard capsules along with additives such as excipients, and the soft capsule preparations may be prepared by mixing the composition according to the present disclosure with the additives such as excipients and then filling the same in capsule bases such as gelatin. The soft capsule preparations may contain, if necessary, plasticizers such as glycerin or sorbitol, colorants, and preservatives.

The definition of the term for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying disease by administering the composition according to the present disclosure. The term “treatment” as used herein refers to any action that improves or favorably changes the symptoms of the disease by administering the composition according to the present disclosure. The term “improvement” as used herein refers to any action that improves the bad state of the disease by administering the composition of the present disclosure to an individual or making the individual intake the composition.

In addition, the present disclosure provides a prevention or treatment method for SARS-CoV-2 infection, including administering, to a subject, a composition including CSNP1, CSNP2, CSNP3 or CSNP4 as an active ingredient in a therapeutically effective amount.

The therapeutically effective amount is an amount effective for treatment of a disease, for example, an amount of a composition administered to a subject to be treated, which may include all amounts of the composition that prevents recurrence, alleviates symptoms, suppresses direct or indirect pathological consequences, prevents metastasis, decelerates a rate of progression, relieves or temporarily alleviates the condition, or improves the prognosis. In other words, the therapeutically effective amount may be interpreted as covering all doses at which symptoms of a disease are alleviated or cured by the composition.

The prevention or treatment method of the present disclosure includes not only dealing with a disease itself before the onset of signs, but also inhibiting or avoiding the signs thereof by administering the composition. In terms of management of a disease, the prophylactic or therapeutic dose of a particular active ingredient will vary depending on the nature and severity of the disease or condition and the route by which the active ingredient is administered. Dose and frequency of dose will vary depending on the age, body weight, and response of an individual patient. A suitable dosage may be easily selected by those of ordinary skill in the art who naturally considers these factors.

### MODES FOR CARRYING OUT THE INVENTION

Hereinafter, examples and example embodiments will be described in detail to help the understanding of the present disclosure. Hereinafter, examples and example embodiments will be described in detail to help the understanding of the present disclosure.

The examples and example embodiments of the present disclosure are provided to more completely explain the present disclosure to those skilled in the art.

### <Examples> Experimental Materials and Methods

The following Experimental Examples are intended to provide Experimental Examples commonly applied to each example embodiment according to the present disclosure.

1. Synthesis of Peptides

Crystalline structures of SARS-CoV-2 spike-RBD that is bound to ACE2 (PDB ID: 6M0J) were used to design CSNP1-CSNP3 peptides. For CSNP4, both a trimer spike-ACE2 (PDB ID: 6ZXN) and an ACE2-RBD complex were considered. For CSNP1-3, hotspot residues of RBD and ACE2 were designated via PDBePISA, and contribution to the interface was evaluated by alanine scanning using DrugscorePPI. A server uses the interface knowledge, calculates the binding energy difference in residues of wild type (AGWT) and mutant (AGMUT) at the interface, and provides hotspot information in terms of numerical values and corresponding 3D b-factor coordinates. RBD that optimally binds to two regions of ACE2 which are a.a. 23-46 and a.a. 352-357 was selected for a design of a mother peptide (CSNP1). Both regions were connected via a GPG loop, and the freedom of K353 was constrained by disulfide bonds (S-S), such that two beta sheets at C350 and C356 loci were stapled.

APBS and APBSrun plug-ins were used to create electrostatic surface maps around hotspots and other interface residues, and a pharmacophore package of MOE (trial version 2019.0102) was used to identify potentially modifiable and important pharmacophores in α1 helix (a.a. 23-46). After determining five potential residues, that are Glu23, Lys26, Thr27, His34, and Gln42 in consideration of surface complementarity, they were substituted to fortify CSNP1-RBD binding using a residue scanning tool in a protein design package of the MOE suit. In the first stage, the mutation window was limited to only one residue, and glycine and automutation were excluded during mutagenesis. Based on binding affinity and stability, the top five mutants were selected, and residue scanning was performed in the second stage to keep the mutation window at 5. Changes in the binding affinity of wild type and mutant peptides based on the binding were double-evaluated via SSIPe and EvoEF. In an attempt to stabilize and maintain the helical structure of the selected peptide, lactam bridges were created at i and i+4 loci of the non-interface residues. For CSNP3, pharmacokinetics in the α1 (a.a. 21-46) region of ACE2 were considered, and a.a. 352-357 regions were excluded. Similar to CSNP1, CSNP4 was designed by being connected to a.a. 445-456 and a.a. 488-501 of SARS-CoV-2 RBD, with no residue scanning implemented. Of the finally selected CSNP peptides, CSNP2-4 were successfully synthesized in 99% (CSNP2), 95% (CSNP3), and 96% (CSNP4) purity identified by reversed-phase high-performance liquid chromatography (HPLC; Shimadzu Prominence) from Peptron Inc. (Daejeon, Korea).

2. Structure of CSNPs Selected Using Molecular Dynamics Simulation

Molecular dynamics simulation is a widely used technique for studying the folding of complicated or isolated forms of proteins and the dynamic behavior of macromolecules. Molecular dynamics simulations were used for structural and dynamic insights of protein-protein, protein-ligand, and protein-DNA/RNA complexes. The basic ABMER99-ILDN force-field was used for simulation of CSNP1, CSNP4, and SBP1 (already identified RBD-binding peptides), and the same force-field was modified for lactam-stapled peptides, CSNP2, and CSNP3. New residues and parameters were modified whenever necessary. The isolated form of peptides was simulated for 200 ns, and simulation was performed for the target binding complex for 100 ns. All simulations were performed in GROMACS 2019.6 with a boundary extended by 10 Å from a protein in a TIP3P water-filled cubic box. All systems were neutralized with the counterion, Na+/Cl−, and energy was minimized under the steepest descending algorithm wherever needed. All systems were then equalized into an NVT ensemble for 0.2 ns and re-equalized into an NPT ensemble for 0.2 ns under constant temperature and pressure, respectively. Temperature and pressure were combined with the V-rescale and Parrinello-Rahman barostat methods, respectively. The bond length was limited by the LINCS algorithm, and the long-range electrostatic interaction was calculated by a particle mesh Ewald algorithm.

3. Calculation of Free Energy Using Molecular Mechanics Poisson-Boltzmann Surface Area

The molecular mechanics Poisson-Boltzmann surface area (MM-PBSA) is a widely used method for calculating relative binding free energy of ligands bound to a target.

Thus, the g_mmpbsa and APBSA tools implemented in GROMACS were used to find calculations of energy that utilize the same MM_PBSA approach. The g_mmpbsa tool is interchangeable with previous version of GROMACS (version 5 or lower), so the “tpr” file created in GROMACS 2019.6 was recreated by GROMACS 5.1 and used to calculate the binding energy. The relative binding energy of the complex was approximated according to the following energy terms:

ΔGbind=ΔEMM+ΔGsol

ΔEMM=ΔEcov+ΔEelec+ΔEvdW

ΔGsol=ΔGpolar+ΔEnon-polar

ΔEMM refers to a change in the gas-phase MM energy, ΔGsol to a change in the solvation free energy, ΔEvdW to a change in the van der Waals energy, ΔEele to a change in the electrostatic energy, and ΔEcov to a change in the covalent energy. The solvation free energy (ΔGsol) was calculated by combining polar and nonpolar energies. All these changes were calculated through an ensemble averaged over a series of forms sampled over the last 25 ns simulation trajectory at time intervals of 0.01 ns.

4. Calculation Tools

Free packages of VMD, Pymol, and Chimera were used for simple visualization and collection of structural insights of SARS-CoV-2 spikes and ACE2 proteins. The sscache.tcl script was used in VMD to monitor the electrostatic surface separation of proteins, APBS, and APBSrun plug-ins in Pymol and VMD and the secondary structural changes of peptides by a time function. VMD was used to create 3D animated videos. The online server PDBePESA (v1.52) and free BIOVIA Discovery Studio Visualizer were used to determine the contribution of each residue for interface analysis and the ACE2-S binding. After using a PPCheck hotspot prediction tool, an alanine scanning package of the same server was used for aniline mutagenesis. The hotspot results were validated via the DrugScorePPI web server, and the results were recorded in terms of energy. The Ligand Scout trial version and the MOE trial version were used for drug group evaluation. However, since our interest was in insolation of amino acid crystalline factors, a drug group model for drug screening was not used. GROMACS 2019.6 was used for molecular dynamics simulation. For MM-PBSA calculations, the “tpr” file created in GROMACS 2019.6 was recreated by GROMACS 5.1 and used for calculation of the binding energy as described above.

5. Surface Plasmon Resonance (SPR)

SPR analysis was performed using Biacore T200 (GE Healthcare, Sweden) technology for physical interaction of CSNP and α1 helix peptide (Pep1-Pep5) and ACE2 and S1 subunits of SARS-CoV-2. The S1 (ligand, AcroBiosystems, S1N-C52H3-100UG, USA) protein was fixed onto a CM5 sensor chip (GE Healthcare, Cat #. BR-1005-30) at a concentration of 6.0 μg/mL using 10 mM sodium acetate (pH 5.5) as a fixing buffer. ACE2 (Acrobiosystems, AC2-C52H7-50 ug, USA) was fixed onto the same chip at a concentration of 6.2 μg/mL using a 10 mM sodium acetate fixing buffer. The following solutions were used as running buffers: 1) HBS (10 mM HEPES, pH 7.4, 150 mM NaCl), 2) HBS-EP (10 mM HEPES, pH 7.4, 150 mM NaCl, 3 mM EDTA, 0.05% P20), and 3) HBS containing 5% DMSO. (10˜50) mM NaOH solution used as a regeneration buffer. CSNPs were injected into a ligand-bound chip at different concentrations, and ka, kd, and KD values were calculated, respectively. A buffer that was acting on was injected into an empty channel as a standard. The experiment was performed twice with a newly prepared reagent, and the data was analyzed in the Software Control (version 2.0.1) and BIAevaluation (version 3.0) software.

6. Immunocytochemical Assay and Confocal Microscope

Human embryonic kidney 293 (HEK293) cells were purchased from Korean Cell Line Bank (KCLB, Chongno, Seoul, Korea) and cultured in a 5% CO₂ humidified incubator in Dulbecco's modified Eagle's Medium (DMEM, Gibco, USA) growth medium containing 10% fetal bovine serum (FBS, Gibco, USA) and 1% antibodies (Gibco, USA). For immunocytochemical assay, HEK293 cells were transfected with 3.0 ug pcDNA3.1-hACE2 (Addgene, 145033, USA) plasmids by Lipofectamine 3000 (Invitrogen, L3000015, USA). Relative amounts of hACE2 were assessed by mRNA expression levels using a primer set (Cosmo genetech, hACE2-F: 5′-TCC ATT GGT CTT CTG TCA CCC G-3′, hACE2-R: 5′-AGA CCA TCC ACC TCC ACT TCT C-3′, Republic of Korea). For CSNPs binding, CE2-overexpressed Hek293 cells were first cultured with 10 uM and 25 10 uM peptides for 1 hour, then treated with 5 uM SARS-CoV-1 S1 protein-His Tag (AcroBiosystems, S1N-C52H3-100UG, USA), and culture for 24 hours. Cells were washed three times with PBS and then cultured at 4° C. for 2 hours in a serum-free medium containing primary antibody anti-ACE2 (1:100, Cell signalling, 4355S, USA), anti-CTNNB1 (1:100, Cell signalling, 8480S, USA), and anti-His-Tag (1:100, Santa cruz, sc-8036, USA). The cells were then fixed with 4% paraformaldehyde for 5 minutes and Alexa Fluor 594 (1:200, Thermo, A21203, USA) and donkey anti-rabbit IgG Alexa Fluor 488 (1:200, Thermo, A21206, USA) were kept at room temperature for 2 hours. Before the secondary antibody was cultured, blocking was performed at room temperature for 1 hour with a cell blocking solution (1% PBS containing 1% BSA and 0.1% Tween 20). All secondary antibodies were diluted with an appropriate concentration of blocking solution. The nucleus was stained with a DAPI-containing mounting solution (Vector, H-1200, USA). The cells were then visualized with the LSM710 (Carl Zeiss) confocal microscope.

7. Analysis on a peptide remaining degree of peptides after ProtK treatment

200 μL of 50 μM stock solution was prepared with a base buffer (10 mM Tris-base, 10 mM NaCl, pH 7.4) and supplemented with 5 μM CaCl₂. For untreated TO samples, 30 μL were obtained from each sample. Thereafter, 5 ug/ml of proteinaseK (Bioshop) was added to a final concentration of the remaining stock. Samples were cultured at 37° C., 30 μL of samples were secured at 10, 30, and 60 minutes respectively, and protease activity was immediately blocked by adding 20 mM PMSF (200 mM stock dissolved in isopropanol). Protease inactivated samples were frozen at −20° C. before further use. The frozen sample was supplemented with 8 μL of sample loading buffer (4×NuPAGE; ThermoFisher Scientific), boiled for 10 minutes (50° C.), and centrifuged at 13,500 RPM for 10 minutes before mounting a gel [12% NuPAGE Bis-Tris (ThermoFisher Scientific)] with 1×Mes running buffer. The gel was treated at 200 V for 35 minutes and stained with Coomassie Brilliant Blue dye G-250 (Thermo Fisher Scientific). ImageJ software having a background subtractive function was used as a density meter. All samples were normalized to untreated samples (TO).

### Example 1. Designing of CSNP

Two strategies were established to neutralize SARS-CoV-2 spikes and design CSNPs to deal with reversible transposition of RBD and S1 masking by soluble ACE2. First, the main scaffold of the α1 helix of ACE2, which is mainly involved in RBD-interactions, was extracted as a starting structure for designing and assembling a helical CSNP (CSNP1-3). Second, the ACE2 interacting motif of RBD was extracted and assembled into CSNP4 to restrict movement of and block the binding to ACE2.

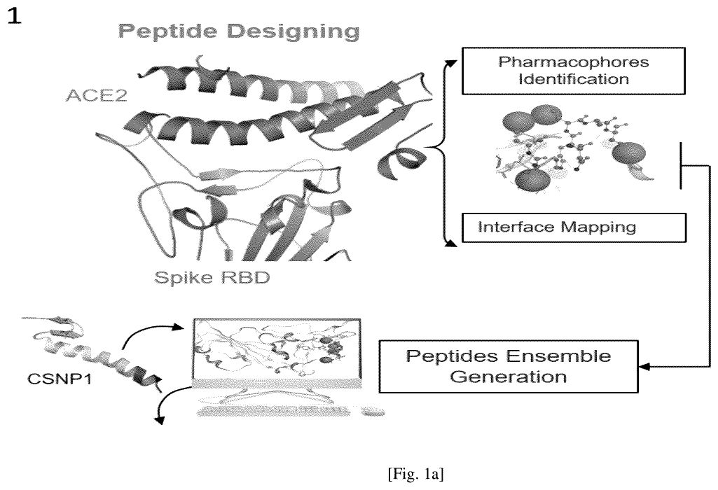

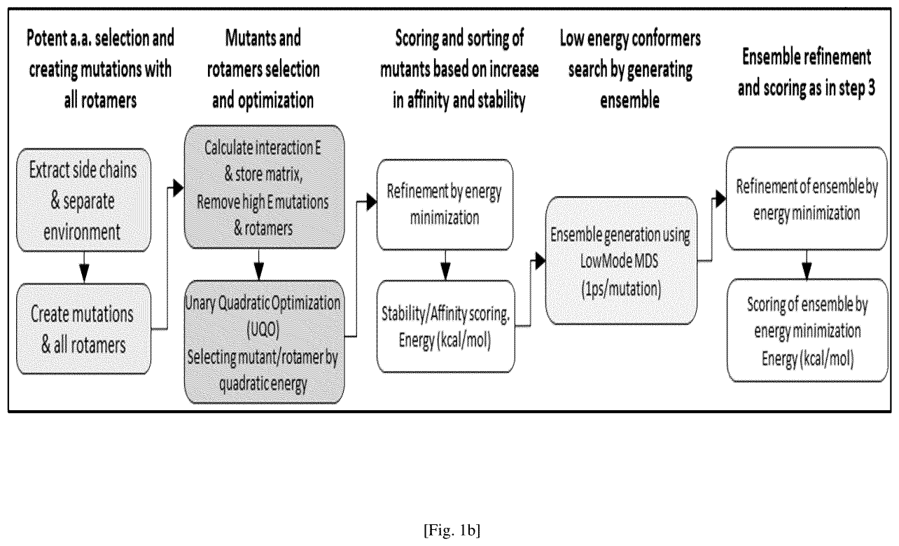

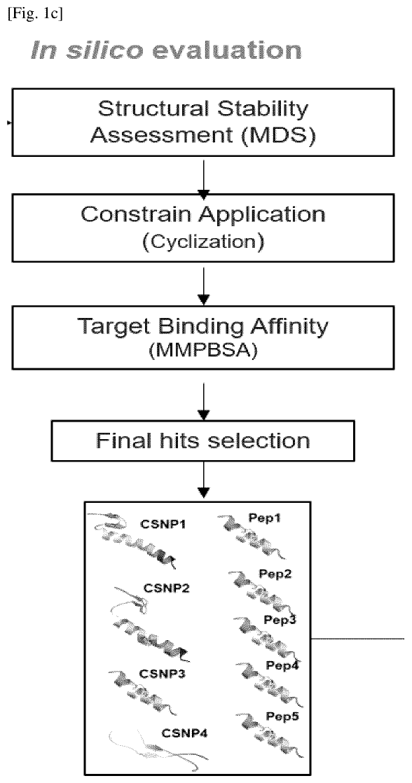

For ACE2, RBD was captured in an upward form through salt bridges and hydrogen bonds mainly using the polar and charged residues of the α1 helix (Table 1). The first three amino acids of al, which are I1e21, Glu22, and Glu23, were exposed to solvents without being involved in RBD bonding. The residues were resulted from the establishment of α1 helix. Five residues, including Gln24, Asp30, Lys31, Asp38, and Tyr41, were found to be the main hotspots of al, contributing to having the highest binding energy in the ACE2-RBD complex. In addition to the α1 helix, ACE2 utilized Lys353 for fixation to the RBD of SARS-CoV and SARS-CoV-2 and shared the second highest binding energy among the ACE2-RBD interface residues. Due to Lys353 placed on the hinge of β3-β4 stapled by disulfide bonds between Cys344 and Cys361, the flexibility and freedom of amino acids may be constrained. This maintained the hydrogen bond network intact between Lys353 of ACE2 and Gly496, Gln498, and Gly502 of RBD (FIG. 1, Table 1). Five drugs such as Asp30, Lys31, Asp38, and Tyr41 in α1 and Lys353 in β3-β4 that keep ACE2-RBD intact were identified. The COOH— group in Asp30 was isolated as a hydrogen bond recipient near the NH3+ group in Lys417 of RBD. The NH3+ group in Lys31 of α1 is located between Glu35 in α1 of RBD and Glu484 of RBD and is an alternately established salt bridge. The COOH— group of Asp38 in α1 is indispensable for stability of Lys353 in ACE2 and also had important contact with Tyr449 and Gln498 in RBD. The bulky side chain in Tyr41 occupies a hydrophobic space between the N-termini of a.a. 350-359 segments and a.a. 21-46 segments in the peptide. The Tyr41-Thr500 hydrogen bonds between ACE2 and RBD constrain rotation of Tyr41. The NH3+ group in Lys353 is a very important drug group with respect to ACE2-RBD interactions (Table 1).

TABLE 1

| ACE2 | RBD | Ekcal/mol | Dist Å |
|---|---|---|---|
| Asp30 | Lys417 | −33.5 | 2.71 |
| Lys353 | Gly496 | −11.1 | 2.71 |
| Lys353 | Gln498 | −7.3 | 2.68 |
| Lys31 | Gln493 | −6.3 | 2.93 |
| Lys31 | Glu484 | −5.64 | 3.24 |
| Glu35 | Gln493 | −4.9 | 2.71 |
| Lys353 | Gly502 | −4.9 | 2.75 |
| Glu37 | Tyr505 | −2.8 | 2.65 |
| Gln24 | Asn487 | −1.9 | 2.81 |
| Tyr83 | Asn487 | −1.7 | 2.64 |
| Gln42 | Gly446 | −1.6 | 2.92 |
| Asp38 | Gln498 | −1.2 | 3.57 |
| Lys31 | Tyr489 | −0.5 | 4.35 |

In the case of the helical peptide, the scaffold of the α1 helix (a.a. 21-46) of ACE2 was cleaved and connected with β3-β4 (a.a. 350-359) via a Gly-Pro-Gly (GPG) linker. The freedom of Lys353 was limited by forming an S—S bond between D350C and F356C loci. The peptides were designated as parent peptides (CSNP1, FIG. 1), and then the complementarity of the electrostatic surface was investigated. This revealed potential points that may improve the binding affinity between CSNP and RBD. Mutations of potential residues Glu23, Lys26, Thr27, His34, and GLN42 consisted of all possible permutations, in consideration of available volume, surface complementarity, total binding energy, and stability of the complex. A database of generated peptides (81 mutants) having a single substitution as well as respective binding affinity and binding stability was recorded and used for the residue scanning in the next round. The top five substitutions of each Glu23, Lys26, Thr27, His34, Gln42 residues were selected and implemented in the multi-substituted peptide forming stage. Monitoring of the binding energy may enable identification of CSNP2 and CSNP2-1 as the best suitable peptides for the RBD interface. To maintain helicity, a structural constraint (lactam bridge) was generated between the side chains of the non-interface residues Phe32Asp and Ala36Lys (FIG. 1). The GPG linker was changed from CNSP2 to PGG to improve flexibility of loop. To verify both the importance of Lys353 for RBD binding and the spontaneity of the α1 helix, shorter constrained peptide, CSNP3, was constructed in consideration of the pharmacological group of the α1 helix.

CSNP4 was designed in consideration of spontaneous transposition as well as ACE2-RBD interface residues of RBD. Two amino acid stretches, 445-456 and 488-501, connected through a long fixed loop participating in ACE2 bonding, were cut off at RBD and bonded via a flexible linker, LIGRGP, to optimally orient the binding peptide and maintain the target binding ability. In the paused (RBDdown) position, the same sheet loop sheet motif lies between the NTD and the RBD domain of the adjusted S protomer. Thus, in addition to ACE2-RBD mutation, it suggests that CSNP4 may constrain spontaneous transposition to represent RBD for immune surveillance.

### Example 2. Structural Stabilization of CSNP

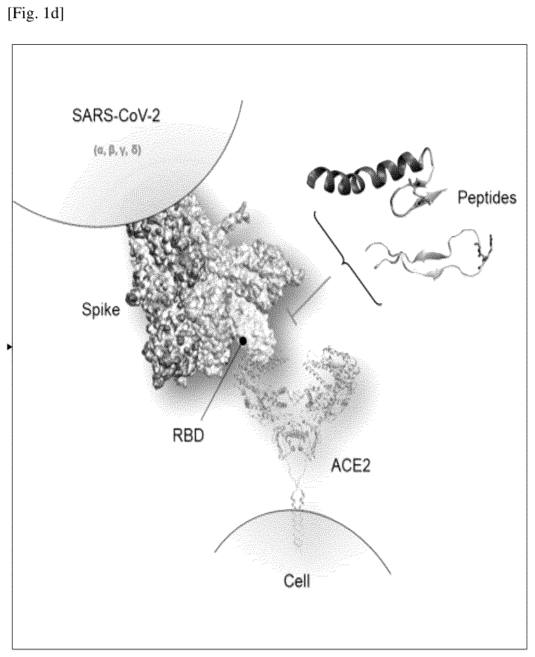

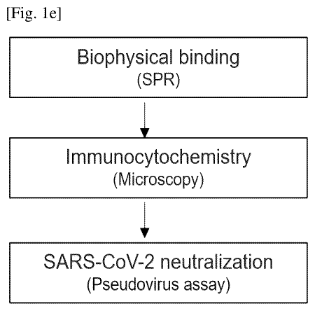

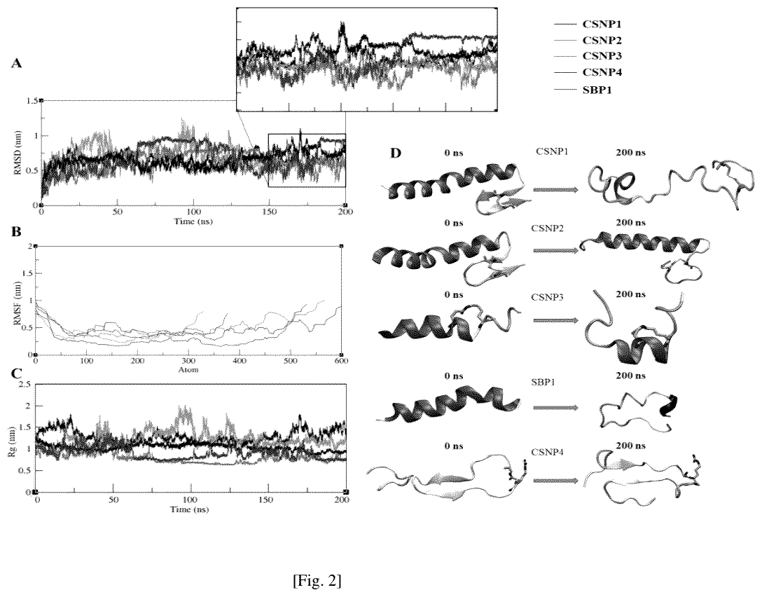

Structural stability and resistance to enzymatic degradation are important features for designing small therapeutic peptides. Moreover, fold-on-binding requires time, and if the structure is not damaged, the peptide often loses target specificity. Thus, the structure of CSNP1-3 was stabilized by applying structural constraint, and the two amino acid stretches of CSNP4 were connected through a shorter loop “LIGRGP”. To determine structural stability, these peptides were simulated in an aqueous environment as a function of time. SBP1, a previously reported RBD binding peptide that is structurally unconstrained and derived from ACE2, was also simulated for validation and stability comparison. Overall, there was a significant root mean square deviation (RMSD) in all atoms on the peptide in the second and third quarters of the simulation (FIG. 2A). To track RMSD variation in the atomic level, root mean square fluctuations (RMSF) for all atoms in all five peptides was calculated. All peptides, especially the terminal atoms of CSNP4, exhibited significant fluctuations compared to that of the peptide body. However, CSNP1 and SBP1 showed high fluctuations in atoms in the range of 100-170 compared to CSNP2 and CSNP3 (FIG. 2B). The rotation radius (Rg) to predict the compression (i.e., folding) of a peptide showed that SBP1 and CSNP3 underwent dramatic changes and exhibited shrinkage of a structure (FIG. 2C). This suggests that the hydrogen bonds between side chains of the peptide probably acquired a new pattern and presumably made the peptide structure disordered. To validate these data, 1000 structural frames were extracted from the 200 ns MD trajectory of each peptide and changes in the secondary structure were investigated. Changes in 3D motion and secondary structure were preserved in the 3D image as a function of time (FIG. 2D). Surprisingly, CSNP1 and SBP1 moved from a helical structure to an irregular loop structure and permanently lost the structural helicity, while CSNP3 partially maintained its helical structure. CSNP2 of the helical peptides maintained its structure intact, but the C-terminal S—S constrained region remained flexible at the PGG junction. The data suggests that it is possible to maintain their target binding affinity to enable stabilization of peptide structures.

### Example 3. Binding Stability of CSNP and Target (RBD and ACE2)

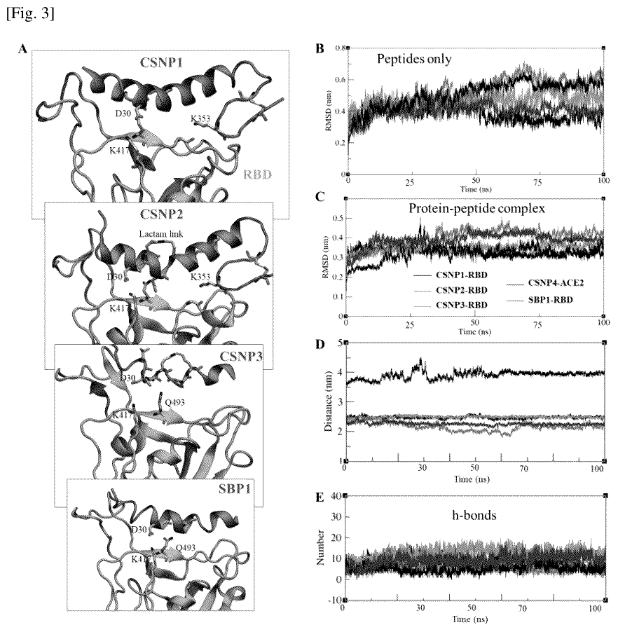

Example 3. Binding stability of CSNP and target (RBD and ACE2) The interface residue of the docked CSNP was identified and observed to overlap with the interface residue of ACE2-RBD (FIG. 3A). It was found that all target binding forms of the peptide were relatively more stable compared to the unbound isolated state (FIG. 3B). CSNP3 had an increase in RMSD due to hydrophilic glutamic acid at the N-terminal, which led to separation of RBD during simulation. It was also found that SBP1 affected the overall RMSD of RBD. Both SBP1 and CSNP3 bound RBD showed similar tendency in RMSD plots (FIG. 3C). To measure dissociation of CSNP from a subject, the average distance between the center of mass and the number of intermolecular hydrogen bonds was calculated as a function of time (FIGS. 3D, E). The average distance between the CSNP1-, CSNP2-, and SBP1-RBD complexes remained constant through the simulation process. However, the distance between CSNP3-RBD and CSNP4-ACE2 remained unstable (FIG. 3D). The distance between ACE2 and CSNP4 was ˜35 nm at the start of the simulation and increased to ˜40 nm at the midpoint (50 ns) of the MD. This increase in distance may be due to the free N- and C-termini of CSNP4, which also had a significant effect on the overall stability (RMSD) of the peptide and the CSNP4-ACE2 complex (FIG. 3B, FIG. 3C). The distance between CSNP3 and RBD fluctuated due to the loosely bound hydrophilic N-terminal glutamic acid of the peptide, which also affected adjacent asparagine and was separated from RBD. Such separation forced the N-terminal of the peptide to perform a whip-like movement. Nevertheless, the C-terminal residue was not damaged with the RBD.

The binding strength between the peptide and the target was estimated using the Poisson-Boltzmann surface area (MM-PBSA) method. The energies for van der Waals (vdW), electrostatic (Ele), polar solvation (PS), and solvent access surface area (SASA) were calculated for all five peptide binding complexes (Table 2). According to the type and length of peptides, they may be divided into three groups. 1) CSNP1 and CSNP2 include both the al helix and the β3-β4 region and bind to RBD. 2) CSNP3 and SBP1 are composed only of al helix and bind to RBD. 3) CSNP4 is completely different from helical peptides and binds to ACE2 and RBD. The overall binding affinity of CSNP1 (total E=−298.44+/−82.0 kcal/mol) and CSNP2 (total E=−283.77+/−81.3) using RBD was relatively similar. However, the energies of vdW and Ele for CSNP2 were stronger than those for CSNP1 (Table 2). Similarly, the total binding energy of the CSNP3 helical peptide (total E=−382.73+/−63.4 kcal/mol) using RBD was relatively stronger than SBP1 (total E=356.73+/−75.1). The polar solvation energy of SBP1-RBD (853.42+/−116.0 kcal/mol) was significantly higher than that of CSNP3-RBD (532.67+/−190.2). The above results suggest that the SBP1-RBD complex may dissociate faster than CSNP3-RBD upon exposure to a solvent.

TABLE 2

| Complex | VDW E | Ele E | PS E | SASA E | Total E |
|---|---|---|---|---|---|
| CSNP1/RBD | −294.49 +/− 26.0 | −685.26 +/− 74.0 | 719.22 +/− 125.0 | −37.91 +/− 3.3 | −298.44 +/− 82.0 |
| CSNP2/RBD | −302.45 +/− 20.3 | −705.56 +/− 96.1 | 763.25 +/− 119.8 | −39.01 +/− 2.4 | −283.77 +/− 81.3 |
| CSNP3/RBD | −225.15 +/− 26.6 | −660.4 +/− 159.4 | 532.67 +/− 190.2 | −29.85 +/− 4.3 | −382.73 +/− 63.4 |
| SBP1/RBD | −282.12 +/− 23.7 | −889.03 +/− 74.0 | 853.42 +/− 116.0 | −39.00 +/− 2.4 | −356.73 +/− 75.1 |
| CSNP4/ACE | −217.30 +/− 29.6 | −1124.5 +/− 72.6 | 422.46 +/− 135.8 | −27.72 +/− 3.4 | −947.06 +/− 104.2 |
| 2 | | | | | |

VDW: van der Waal;
Ele: Electrostatic;
PS: Polar Solvation;
SASA: Solvent accessible surface area;
E: Energy.
All energies are measured in kcal/mol.

### Example 4. Effects of CSNP on S1 Binding to hACE2-Expressing Cells

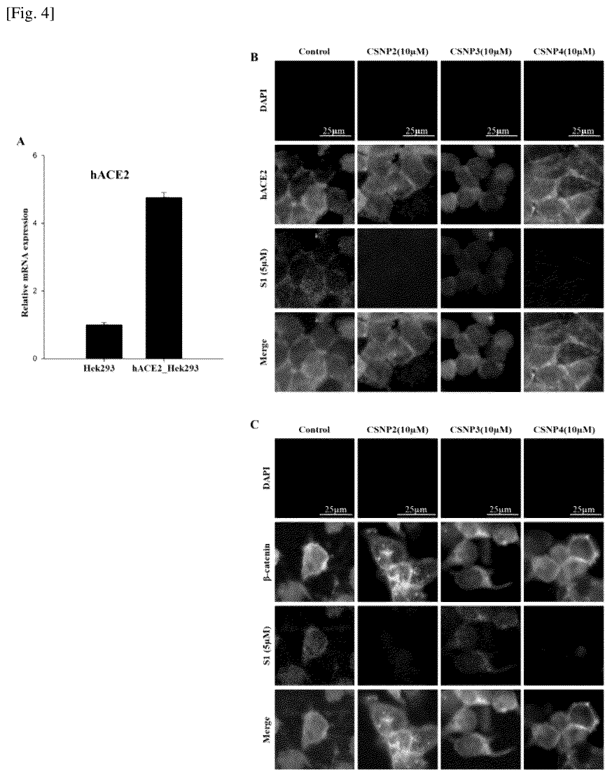

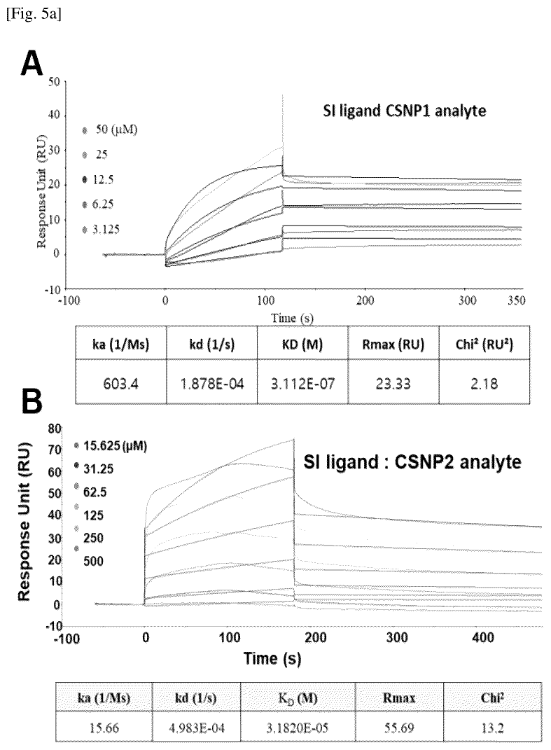

Human ACE2 (hACE2)-overexpressing cells were prepared by transfecting HEK293 cells with hACE2-expressing plasmid, pcDNA3.1-hACE2, and treated with peptides (FIG. 4A). It was shown that CSNP3 completely eliminated the S1-ACE2 interaction while S1 was localized to the cell membrane in CSNP untreated cells (FIG. 4B). In order to localize S1 on the cell membrane and further determine an inhibitory effect of the peptide, experiment was repeated in hACE2-HEK cells labeled with β-catenin. It was found that CSNP2 and CSNP4 completely blocked the membrane localization of S1 (FIG. 4C).

### Example 5. Biophysical Interaction of CSNP and Target Proteins (SPRs)

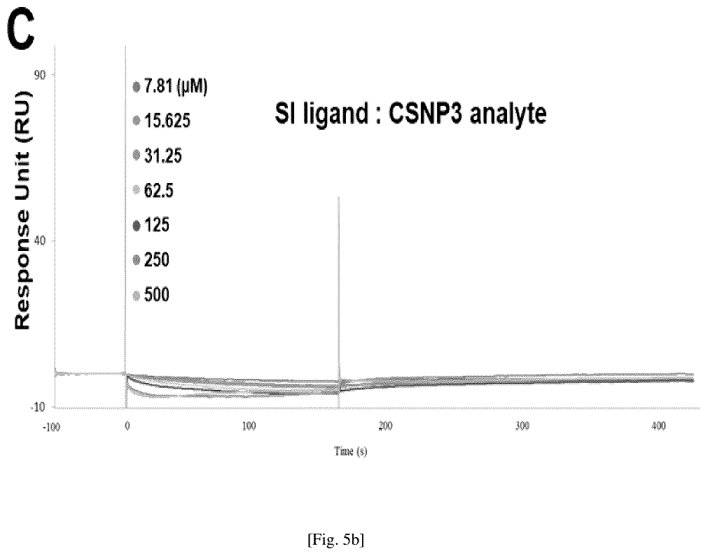

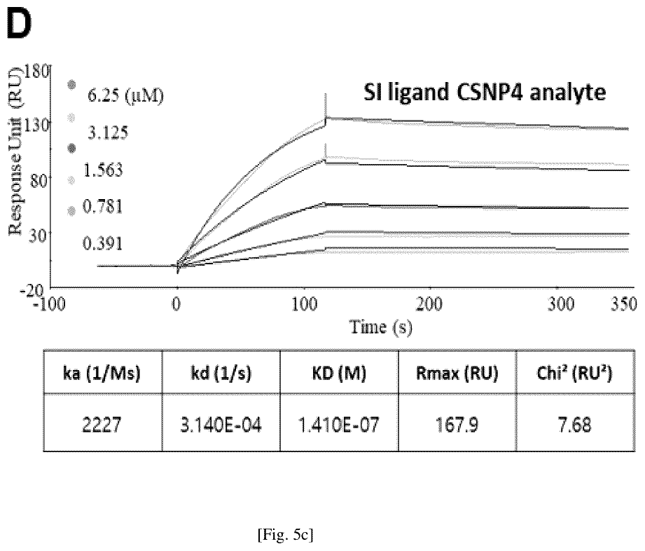

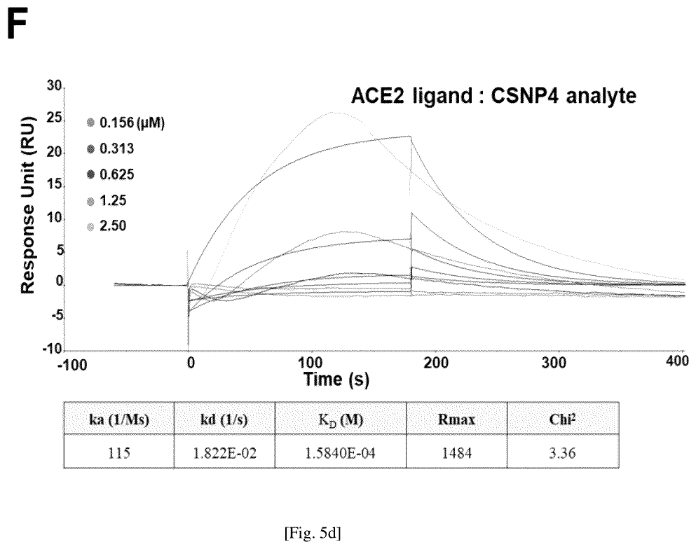

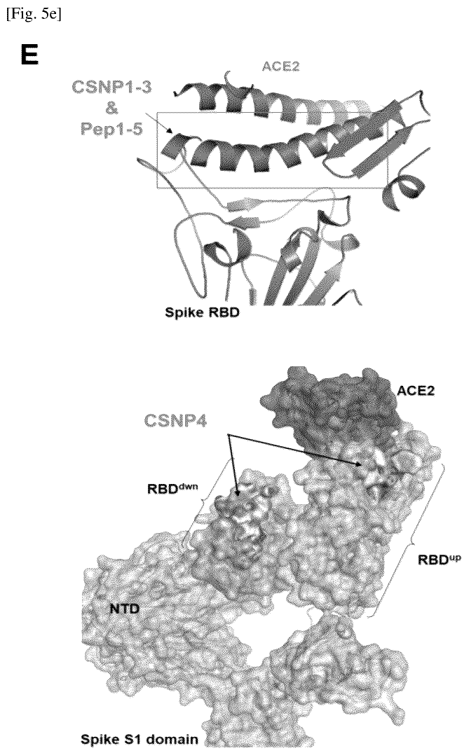

SPR is considered a reproducible and sensitive technique compared to biolayer interferometers, which is to observe the binding dynamics and biophysical interactions of CSNP and targets. As a verification stage, ACE2 and S1 subunits were fixed with ligands onto the CM5 sensor chip and tested for each type of CSNPs (analytes). When S1 was used as the ligand and CSNP1 as the analyte, KD was 0.3 μM (KD=0.3 μM), showing dependent binding (FIG. 5A).

CSNP2 and CSNP3 were injected as analytes in S1 immobilized at 6 and 7 concentrations, respectively. CSNP2 exhibited dose-dependent binding to the S1 protein with KD of 31.8 μM (KD=31.8 μM) (FIG. 5B), and CSNP3 showed no dependent binding (FIG. 5C). The results suggest that CSNP2 and probably CSNP1 maintained the binding to the RBD. In addition, CSNP4 also showed dependent binding with KD of 0.3 μM (KD=0.3 μM) (FIG. 5D).

As a result of measuring the binding dynamics of CSNP4 and fixed ACE2, a relatively weak and dose-dependent binding affinity was shown (KD=158 μM) (FIG. 5F). The binding affinity of CSNP4 and S1 was estimated in view of the immunocytochemical results (FIG. 4) and the fact that CSNP4 occupies the junction between NTD and RBDup of adjacent S protomer in the trimer spike protein (FIG. 5F). It was shown that CSNP4 binds to S1 as strong as CSNP2 (FIG. 5D). The weaker binding affinity of CSNP4-ACE2 is explained for the following two reasons. First, the α1 helix of ACE2 provides a narrow and shallow surface for CSNP4 binding. Second, Lys417 (Table 1), which significantly contributes to ACE2-RBD binding, is not included in CSNP4. The results suggest that designing of an RBD-derived bait peptide for ACE2 may not be sufficient to block ACE2-RBD interactions due to the wide stereoscopic space and irregular loop structure of the ACE2-binding motif in RBD. CSNP4-based S1 neutralization may be attributed to stronger CSNP4-S1 binding, which may constrain the freedom of RBD and ultimately bind to ACE2.

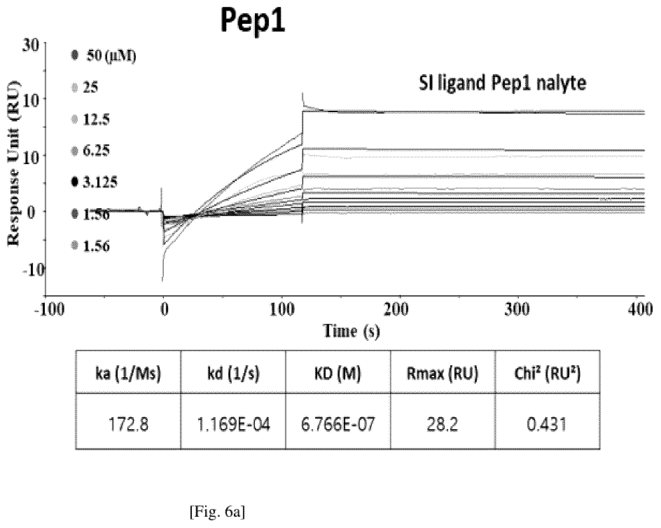

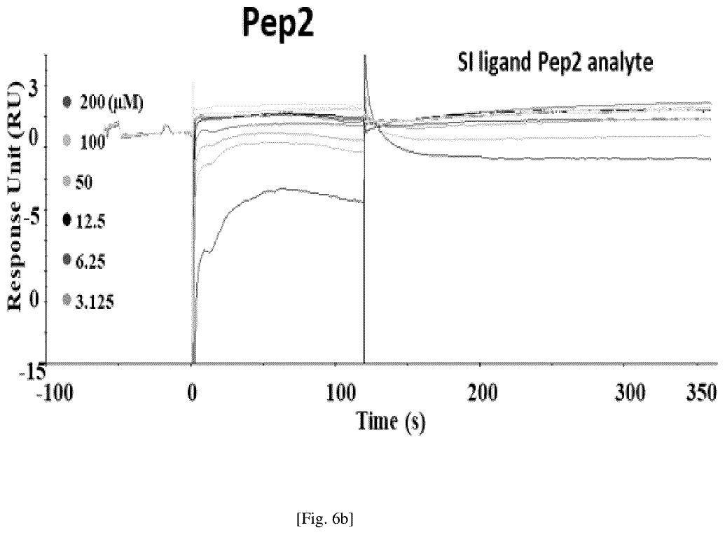

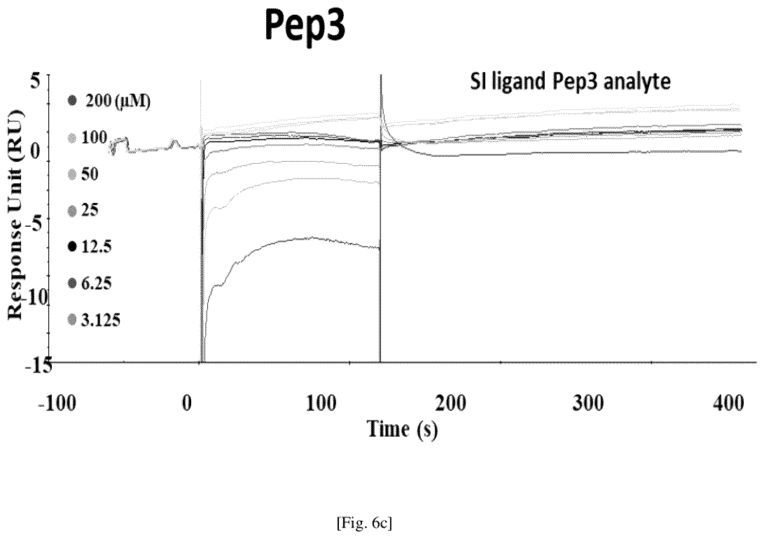

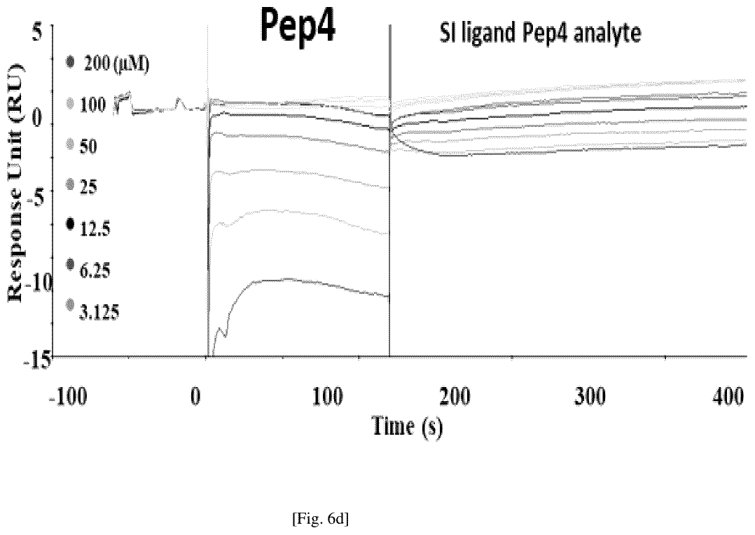

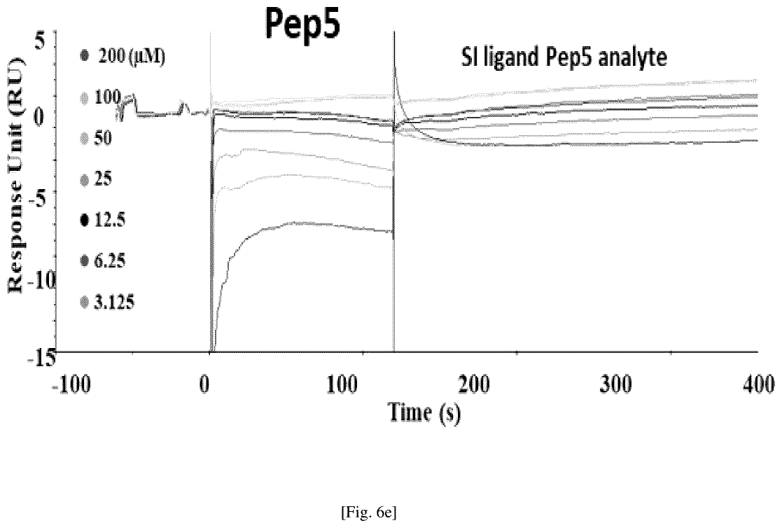

In addition, when S1 was used as a ligand and α1 helix peptide (Pep1˜Pep5) as an analyte, Pep 1 showed dependent binding with KD of 0.67 μM (KD=0.67 μM), while the remaining peptides did not show dependent binding (FIG. 6).

### Example 6. Enzymatic Stability of Structurally Unnatural Linear Peptides

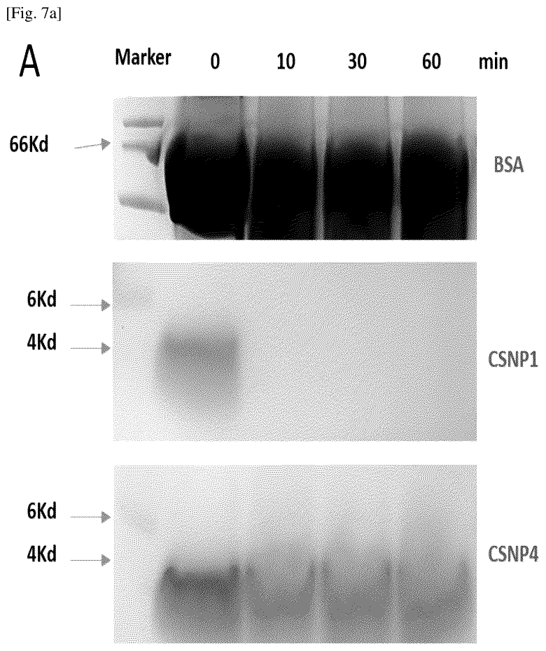

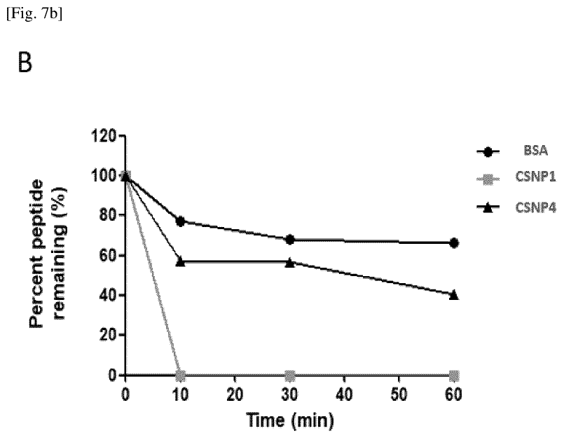

As a result of analyzing the peptide remaining degree after treating the peptide with proteinase K and staining, it was found that CSNP4 was remained in a certain amount, while CSNP1 was barely remained (FIG. 7).

As described above, a specific part of the content of the present disclosure is described in detail, for those of ordinary skill in the art, it is clear that the specific description is only a preferred embodiment, and the scope of the present disclosure is not limited thereby. In other words, the substantial scope of the present disclosure may be defined by the appended claims and their equivalents.

1. A pharmaceutical composition for prevention or treatment of SARS-CoV-2 infection, comprising CSNP1, CSNP2, CSNP3, or CSNP4 as an active ingredient.

2. The pharmaceutical composition of claim 1, wherein the CSNP1 has an amino acid sequence represented by SEQ ID NO: 1, the CSNP2 has an amino acid sequence represented by SEQ ID NO: 2, the CSNP3 has an amino acid sequence represented by SEQ ID NO: 3, and the CSNP4 has an amino acid sequence represented by SEQ ID NO: 4.

3. The pharmaceutical composition of claim 1, wherein the CSNP1, CSNP2, CSNP3, or CSNP4 inhibits interaction of a spice protein of SARS-CoV-2 with ACE2.

4. The pharmaceutical composition of claim 1, wherein the CSNP1, CSNP2, CSNP3, or CSNP4 binds to a receptor binding domain (RBD) of a spice protein of SARS-CoV-2.

5. The pharmaceutical composition of claim 1, wherein the CSNP2 and CSNP3 have lactam rings.

6. A co-administration composition for prevention or treatment of SARS-CoV-2 infection, comprising CSNP1, CSNP2, CSNP3, or CSNP4 as an active ingredient.

7. A health functional food composition for prevention or alleviation of SARS-CoV-2 infection, comprising CSNP1, CSNP2, CSNP3, or CSNP4 as an active ingredient.

8. A prevention or treatment method for SARS-CoV-2 infection, comprising administering, to a subject, a composition including CSNP1, CSNP2, CSNP3 or CSNP4 as an active ingredient in a therapeutically effective amount.


SEQUENCE LISTING (4 sequences)

SEQ ID NO: 1 - protein, 40 aa, Artificial Sequence
  CSNP1
IEEQAKTFLDKFNHEAEDLFYQSSLASGPGCLGKGDCRIL

SEQ ID NO: 2 - protein, 38 aa, Artificial Sequence
  CSNP2
LQAANFLDKDNDEKEDLFYKSSLASPGGCLGKGDCRIL

SEQ ID NO: 3 - protein, 20 aa, Artificial Sequence
  CSNP3
ENAINFLDKDNHEKEDLFYQ

SEQ ID NO: 4 - protein, 32 aa, Artificial Sequence
  CSNP4
WGQNYNYLYRLFLIGRGPCYFPLQSYGFQPTN